(19) United States
(12) Patent Application Publication
(10) Pub. No.: US 2023/0360329 A1
(43) Pub. Date: Nov. 9, 2023

(54) Metaverse Experiences

(71) Applicant: Meta Platforms Technologies, LLC, Menlo Park, CA (US)

(72) Inventors: Matthew Joseph ESQUIVEL, Redmond, WA (US); Alexander Irvin HOPMANN, Seattle, WA (US); Zimran Derkesen AHMED, Palo Alto, CA (US); David PICKHAM, Menlo Park, CA (US); Myriam AMSALLEM, San Francisco, CA (US); John RUMSFELD, San Francisco, CA (US); Wen LI, Foster City, CA (US)

(73) Assignee: Meta Platforms Technologies, LLC, Menlo Park, CA (US)

(21) Appl. No.: 18/352,439

(22) Filed: Jul. 14, 2023

Related U.S. Application Data
(60) Provisional application No. 63/382,153, filed on Nov. 3, 2022.
(60) Provisional application No. 63/371,335, filed on Aug. 12, 2022.
(60) Provisional application No. 63/390,357, filed on Jul. 19, 2022.

(51) Int. Cl. G06T 19/00; G06F 9/54; A61B 5/00; A61B 5/11

(52) U.S. Cl. CPC G06T 19/00; G06F 9/547; A61B 5/4866; A61B 5/1116; G06F 40/197

(57) ABSTRACT

In some implementations, the disclosed systems and methods display content from a public network (e.g., the Internet) such as via a web host, content delivery network, etc. In some implementations, the disclosed systems and methods can issue an entitlement as a token, signed with a private key by an issuing authority. In some implementations, the disclosed systems and methods can determine caloric consumption attributable to both upper and lower body movements of the user throughout changes in pose.

### CROSS REFERENCE TO RELATED APPLICATIONS

This application claims priority to U.S. Provisional Application No. 63/390,357 filed Jul. 19, 2022 and titled “Dynamically Displaying Three-dimensional Web Content to a User,” 63/371,335 filed Aug. 12, 2022 and titled “Decentralized Entitlements,” and 63/382,153 filed Nov. 3, 2022 and titled “Automatic Energy Expenditure Estimation in Artificial Reality.” Each patent application listed above is incorporated herein by reference in their entireties.

### BACKGROUND

Display systems can display content to users in a variety of formats that match user preferences or use cases. However, content can have a variety of display configurations, and effectively displaying content in accordance with user selections and/or expectations remains a challenge. For example, web content can be configured according to the display capabilities of a client system, however friction points in the correspondence between the client system and a web host result in content that does not fully leverage the client system display capabilities or that fails to render in a manner that meets user expectations.

An entitlement can be a right to use, access, or consume a resource, such as an application (e.g., via a temporary or permanent license), an item (e.g., a virtual object, a virtual world, an avatar, etc.), and the like. An entitlement can also be referred to as an authorization, access right, permission, rule, and/or privilege with respect to resource. Conventionally, an entitlement is tied to a particular authority and can only be used with respect to that authority. For example, a virtual item tied to a particular artificial reality (XR) application can typically only be used within that application.

Interactions with artificial reality systems can be fast-paced, requiring highly dynamical changes in positioning (i.e., pose) for an operating user. Such changes can result from the user striving to complete certain actions that can be associated with accomplishing one or more goals for a particular artificial reality environment. Often such actions involve both upper and/or lower body movements. For instance and where lower body movements are involved, the same can entail walking, running, lunging, squatting, etc.

In light of growing societal interest to evaluate how any one particular activity can contribute to improving an individual's health, such as by exercise, finding new ways to provide such an evaluation even in the context of artificial reality can be attractive. That is, providing an accurate accounting of caloric expenditure of a user occurring during that user's experience in an artificial reality environment can be informative to the user for evaluating her health and fitness goals.

### SUMMARY

Aspects of the present disclosure are directed to an artificial reality browser configured to dynamically display three-dimensional web content. A web browser can display content from a public network (e.g., the Internet) such as via a web host, content delivery network, etc. The artificial reality browser can be configured to display, via a client system, both two-dimensional (2D) web content and three-dimensional (3D) web content. In some implementations, communication between the web browser (e.g., native browser code) and web code that implements the webpage can trigger coordinated: a) display transitions (e.g., from a 2D mode to a 3D mode) at the client system that displays the web browser; and b) content transitions such that 3D content can be generated by the web code and/or web source. Implementations achieve the coordination using an application programming interface (API) exposed by the web browser and API call(s) from the web code.

Further aspects of the present disclosure are directed to decentralizing entitlements, i.e., rights with respect to virtual items. Implementations can issue an entitlement as a token, signed with a private key by an issuing authority. Implementations can embed the token with features of the entitlement, providing all data needed to move the token outside of the issuing authority. A validating authority different than the issuing authority can validate and grant the entitlement by checking, using a public key associated with the issuing authority, that the signature on the token was from the issuing authority, without needing the issuing authority to validate the token. Implementations can thus enable entitlements to be decentralized, so that they can be used across multiple systems. Implementations can issue the token to a messaging account associated with a recipient user, eliminating the need for a separate virtual wallet.

Yet further aspects of the present disclosure are directed to estimating a user's energy expenditure during that user's interactions with an artificial reality system. For the estimation, an energy expenditure calculation system can determine caloric consumption attributable to both upper and lower body movements of the user throughout changes in pose. For one or more changes in user lower body posture, the energy expenditure calculation system can assess an amount of caloric expenditure due to a change in user positioning measured for certain physiological attributes of the user. As a result, such an expenditure can then be combined with an associated expenditure for the user's upper body movements to reach a complete energy expenditure estimation.

### BRIEF DESCRIPTION OF THE DRAWINGS

FIG. 1 illustrates an example web browser that display two-dimensional web content.

FIG. 2 illustrates stereoscopic versions of an example web browser that displays stereoscopic web content.

FIG. 3 illustrates a user view of an example web browser that displays three-dimensional web content.

FIG. 4 illustrates an example system diagram for dynamically displaying three-dimensional web content to a user.

FIG. 5 is a flow diagram illustrating a process used in some implementations of the present technology for dynamically displaying three-dimensional web content to a user.

FIG. 6 is a flow diagram illustrating a process used in some implementations for issuing decentralized entitlements by an issuing authority.

FIG. 7 is a flow diagram illustrating a process used in some implementations for granting decentralized entitlements by a validating authority.

FIG. 8A is a conceptual diagram illustrating an example token as a readable text block in accordance with some implementations of the present technology.

FIG. 8B is a conceptual diagram illustrating an example encoded token in accordance with some implementations of the present technology.

FIG. 8C is a conceptual diagram illustrating an example Quick Response (QR) code representing a token in accordance with some implementations of the present technology.

FIG. 9 is a conceptual diagram illustrating an artificial reality system user disposed in a first pose.

FIG. 10 is a conceptual diagram illustrating an artificial reality system user disposed in a second pose.

FIG. 11 is a conceptual diagram illustrating an example kinematic model of a user.

FIG. 12 is a flow diagram illustrating a process used in some implementations for determining a total energy expenditure of a user due to movements for an artificial reality environment.

FIG. 13 is a block diagram illustrating an overview of devices on which some implementations of the present technology can operate.

FIG. 14 is a block diagram illustrating an overview of an environment in which some implementations of the present technology can operate.

### DESCRIPTION

Aspects of the present disclosure are directed to an artificial reality browser configured to dynamically display three-dimensional content. A web browser can display content from a public network (e.g., the Internet) such as via the host of a webpage, a content distribution network, or any other network source of web content. The artificial reality browser can be configured to display, via an artificial reality client system, both two-dimensional (2D) web content and three-dimensional (3D) web content. In some implementations, communication between the web browser (e.g., native browser code) and web code that implements the webpage can trigger coordinated: a) display transitions (e.g., from a 2D mode to a 3D mode) at the client system that displays the web browser; and b) content transitions such that 3D content can be generated by the web code and/or web source.

In some implementations, the coordination between the artificial reality browser and the web code/web source can be achieved via one or more application programming interface (API) calls. For example, the artificial reality browser can expose an API to the web code that implements the webpage. In some implementations, a first API call from the web code/web source can request device capabilities of the client system that runs the artificial reality browser and/or display capabilities of the artificial reality browser itself, such as whether the client system and artificial reality browser are capable of displaying 3D content. The artificial reality browser can respond to the first API call with an indication of the display capabilities, such as ‘True’ or ‘False’ (e.g., 1 or 0).

Once the web code/web source determines that the client system is capable of rendering 3D content, the web code/web source can issue a second API call that initiates a display transition at the client system. For example, the second API call can indicate to the artificial reality browser that the web code will generate 3D version(s) of the webpage (e.g., stereoscopic versions) for rendering in 3D. The second API call can create an asynchronous promise (e.g., JavaScript asynchronous promise) that is fulfilled when the 3D version(s) of the webpage are received at the artificial reality browser and/or when supporting resources are loaded.

In some implementations, after transmitting the second API call, the web code can generate the 3D version(s) of the webpage, such as two stereoscopic versions that are configured to be rendered at each display of an artificial reality client system (e.g., to each eye of a user) to achieve a 3D user view of the webpage. Once the 3D version(s) of the webpage are generated and/or the resources that support the 3D version(s) are loaded, the asynchronous promise can be met and at least portions of the webpage can be rendered to the user in 3D using a 3D mode of the artificial reality client system. For example, at least portions of web content in each of the stereoscopic versions of the webpage can be positioned and configured such that the web content is perceived with depth (e.g., in 3D) when viewed by the user.

Implementations generate stereoscopic versions of the webpage using any suitable techniques. For example, logic present in the webpage/web code can be triggered to generate stereoscopic versions of the webpage (e.g., when the response to the first API call indicates that the browser/artificial reality system are capable of 3D display). In some implementations, the 3D mode for an artificial reality browser can split a viewport (e.g., horizontally, vertically, etc.) and the stereoscopic versions of the webpage can be displayed at each side of the split viewport. In an example, the web code can use canvas element(s) (e.g., an HTML5 canvas element) to selectively draw stereoscopic content in the stereoscopic versions of the webpage. In another example, the web code can trigger the placement of stereoscopic versions of a full screen video.

In some implementations, tags or styles (e.g., CSS class tags, CSS styles, etc.) can be used to selectively position and configure content in the stereoscopic versions of the webpage such that the content is perceived with depth when a user views the content via an artificial reality system. For example, JavaScript logic and/or CSS tags can be used to duplicate a webpage into two versions and reposition content items within the two versions to support stereoscopic display of the two versions via an artificial reality system. In some implementations, the web code logic can trigger content item substitutions such that stereoscopic versions of content items (e.g., images, videos, etc.) are substituted in the stereoscopic versions of the webpages.

In another example, DOM trees can represent two stereoscopic versions of the webpage, the versions positioning and configuring one or more content items such that, when a user views the content items via an artificial reality system, the content is perceived with depth (e.g., in 3D). For example, classes at the root of both DOM trees can correspond to specific stereoscopic versions (e.g., left eye and right eye) and transformations can select these classes to position and configure the content. In another example, an HTML header tag (or any other suitable HTML tag) can define the two stereoscopic versions of the webpage so that content, when viewed by a user via an artificial reality system, is perceived with depth. For example, web code (e.g., JavaScript, PHP, etc.) can trigger the generation of HTML tag(s) that position and configure the webpage content.

In some implementations, the entirety of a webpage is positioned and configured so that the webpage, when viewed by a user via an artificial reality system, is perceived with depth in the stereoscopic versions. In other implementations, select content is positioned and configured such that the content is perceived with depth when viewed by a user via an artificial reality system in the stereoscopic versions. For example, content comprising a predefined tag, attribute, and/or property can cause the content to be selectively positioned and configured to cause a 3D effect when viewed by a user via an artificial reality system.

In some implementations, the content provided by the source can be dynamic itself such that, when the artificial reality system displays the web browser in a 2D mode, a 2D version of the web page is displayed and, when the artificial reality system displays the web browser in a 3D mode, a 3D version of the web page is displayed. For example, web code can be configured to detect display parameters, such as a display mode for the artificial reality browser, display hardware (e.g., two coordinated display devices), a split viewport, etc. In some implementations, logic in the web code (e.g., JavaScript, PHP: Hypertext Preprocessor (PHP), CSS media query, etc.) can detect when the hardware and/or artificial reality browser are in a state to display stereoscopic versions of a webpage (e.g., are in 3D mode). For example, a CSS media query can trigger stereoscopic versions of the webpage when the query returns that the webpage is being displayed in a 3D capable manner (e.g., a head-mounted display of an artificial reality system). In another example, the artificial reality system can expose an API that can return (to the web code) the display state for the device (e.g., whether the artificial reality system is in 3D mode).

The web code can trigger generation of the stereoscopic versions of the webpage (for rendering) based on this detection. Any suitable technique can be used to generate the stereoscopic versions of the webpage upon detection that the client system and/or artificial reality browser are in a state to display 3D content. When the logic in the web code detects that the client system and/or artificial reality browser are not in a state to display 3D content (e.g., not in 3D mode) the logic can generate a traditional 2D webpage for display at the artificial reality browser/artificial reality system.

In some implementations, when the artificial reality browser is transitioned to a 3D mode by web code (e.g., based on an API call that indicates stereoscopic versions of a web page will be rendered), interface elements of the artificial reality browser itself can also be rendered in 3D. For example, transitioning the artificial reality browser to 3D mode can cause menu(s), menu item(s), button(s), and other suitable elements of a browser chrome to render in 3D (e.g., render with a positioning and configuration such that a user perceives the elements of the browser with depth).

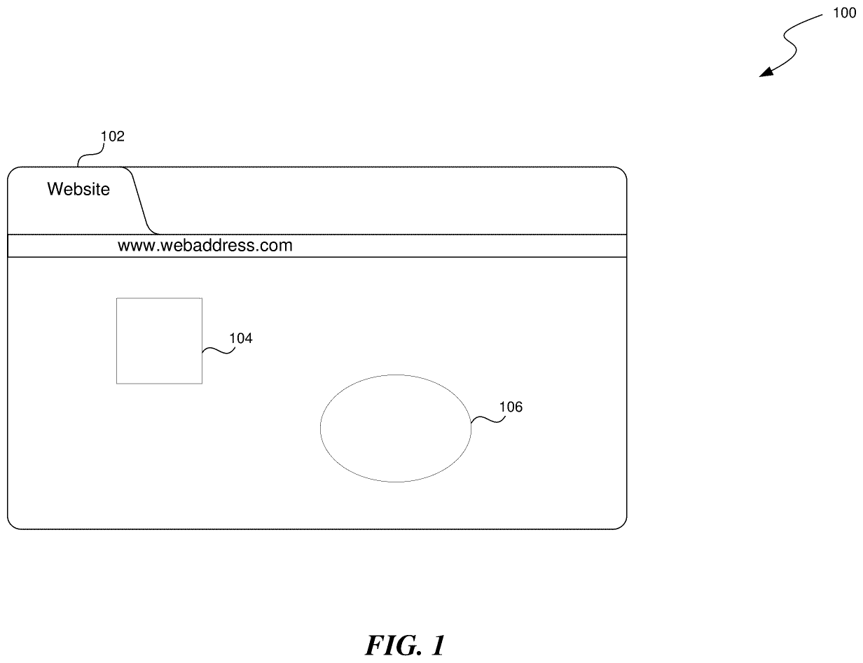

FIG. 1 illustrates an example web browser that display two-dimensional web content. Diagram 100 illustrates browser 102 that displays a website with content components 104 and 106. For example, content components 104 and 106 can be rendered in 2D. In some implementations, browser 102 can be an artificial reality (XR) browser capable of rendering content in both 2D and 3D. For example, the XR browser can have a 2D mode, where standard web content is displayed in 2D, or a 3D mode, where stereoscopic versions of a webpage display web content in 3D. In some implementations, the 2D mode and 3D mode can be implemented by a client system, such as an XR system with a head-mounted display (HMD). In the illustrated example, browser 102 can be an XR browser in a 2D mode.

Implementations of an XR browser can display received/retrieved/stored web content to the user via the XR system. For example, the XR browser can load web resources from a web host, content distribution network, cache, or any other suitable source of web content. In some implementations, the XR browser can include a panel window that displays two-dimensional or three-dimensional web content, as depicted by browser 102.

In some implementations, an XR browser can run at least two portions of code. A first code portion can be browser code that implements functionality for the XR browser itself and a second code portion can be web code that implements webpage functionality. Implementations of the browser code can be first-party code (e.g., native code at the user system, cloud-based code for the XR browser, or other suitable first-party code) and implementations of the web code can be third-party code (e.g., code received/retrieved from a web source). In some implementations, web code can be client-side web code (e.g., run at the XR browser) or server-side web code (e.g., run at the web host or other suitable computing device). In some implementations, the browser code can expose an API to the web code such that API call(s) from the web code can be received by the XR browser, for example to trigger transition of a display mode for the XR browser and/or signal transition of webpage versions (e.g., from standard 2D version to stereoscopic versions). Disclosure about API calls from the web code to the XR browser are further described below.

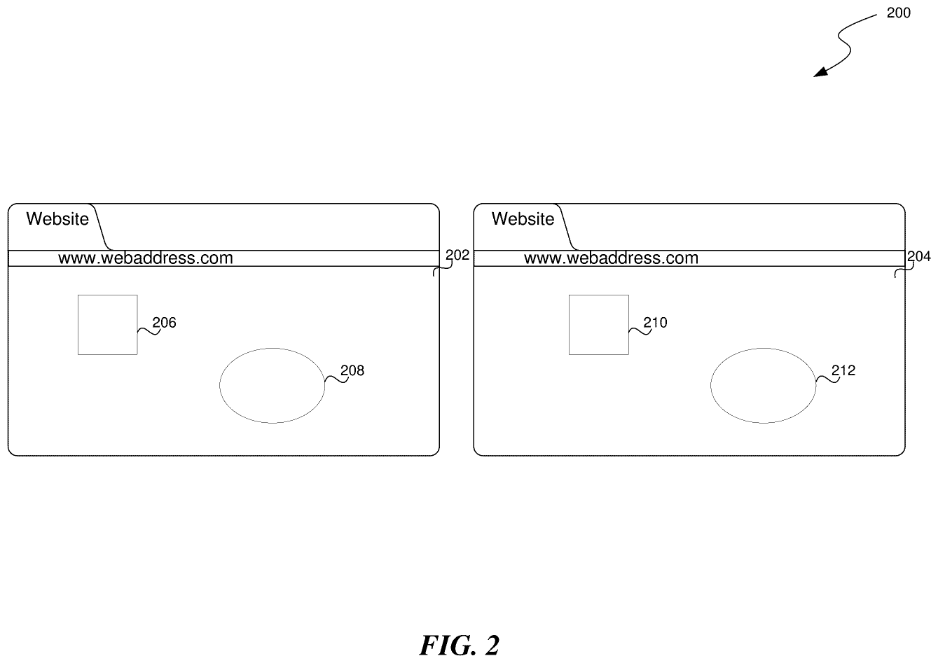

In some implementations, once a 3D mode is triggered for an XR browser, multiple versions of a webpage can be provided to the XR browser for display, such as stereoscopic versions. FIG. 2 illustrates stereoscopic versions of an example web browser that displays stereoscopic web content. Diagram 200 illustrates stereoscopic webpage versions 202 and 204 displayed at an XR browser with stereoscopic content components 206, 210, 208, and 212. In some implementations, stereoscopic version 202 of the web page can be configured to be displayed to a user's left eye (or can be configured as the top stereoscopic version of the web page) and stereoscopic version 204 of the web page can be configured to be displayed to a user's right eye (or can be configured as the bottom stereoscopic version of the web page). For example, in 3D mode, the XR browser can split the viewport of the browser to support display of the two stereoscopic versions of the webpage.

In some implementations, stereoscopic content components 206 and 210, contained in stereoscopic webpage versions 202 and 204, respectively, can be positioned in coordination such that the content components are perceived with depth when webpage versions 202 and 204 are displayed to a user (e.g., via an HMD of a XR system). For example, one or more transformation (e.g., implemented via CSS class tags, CSS styles, HTML, JavaScript, etc.) can position and configure content components 206 and 210 at locations that are perceived as three-dimensional when viewed by the user (e.g., when stereoscopic webpage version 202 is viewed by a left eye of the user and stereoscopic webpage version 204 is viewed by the right eye of a user). Similarly, stereoscopic content components 208 and 212 can be positioned in coordination such that the content components are perceived as three-dimensional when webpage versions 202 and 204 are displayed to a user.

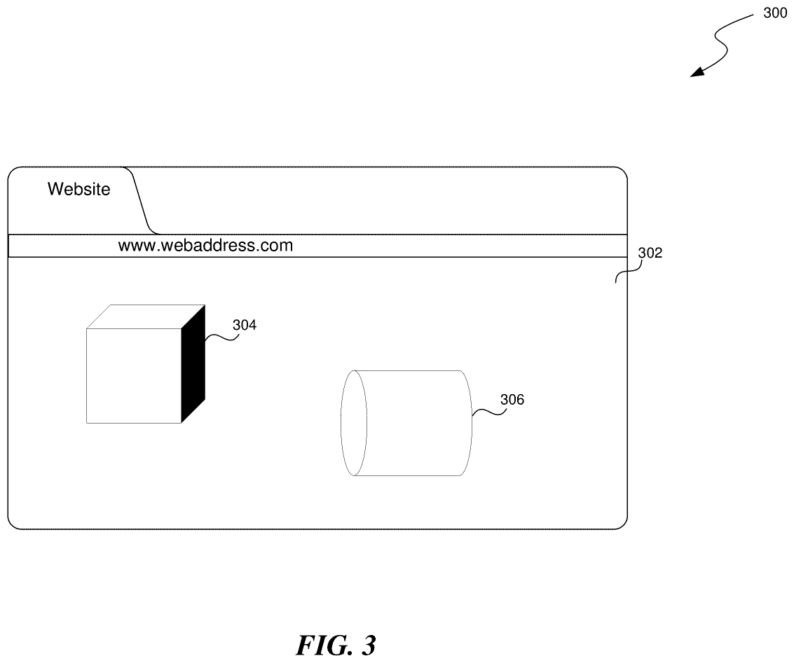

FIG. 3 illustrates a user view of an example web browser that displays three-dimensional web content. Diagram 300 illustrates a user view of 3D webpage 302 in an XR browser (e.g., XR browser in a 3D mode) and a user view of 3D content components 304 and 306. For example, 3D webpage 302 can be the user view of stereoscopic web pages 202 and 204 of FIG. 2, such as the user's perception when stereoscopic web pages 202 and 204 are displayed to the user by an XR system. Similarly, 3D content component 304 can be the user view of stereoscopic content components 208 and 212, and 3D content component 306 can be the user view of stereoscopic content components 208 and 212.

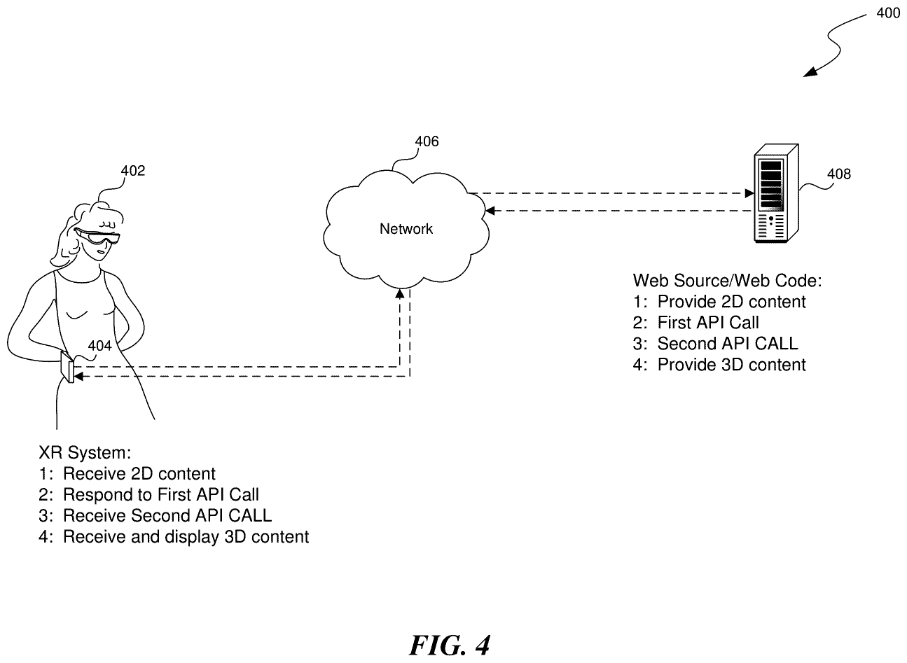

In some implementations, coordination between web code and the XR browser can be accomplished using one or more API calls. FIG. 4 illustrates an example system diagram for dynamically displaying three-dimensional web content to a user. System 400 includes user 402, XR system 404, network 406, and web source 408.

In some implementations, XR system 404 can display webpages/web content to user 402 via a web browser (e.g., XR browser). The web browser can receive or retrieve the webpages/web content from web source 408 (e.g., a content delivery network, host server, cache, or other suitable web source). Traffic between XR system 404 and web source 408 can be carried over network 406 (e.g., the public Internet).

In the illustrated implementation, web source 408 provides web code that is loaded at run at the web browser at XR system 404. For example, the web code provides 2D content to XR system 404, coordinates with XR system 404 using API call(s), and transitions to providing 3D content to XR system 404. For example, XR system 404 can receive and render 2D content via a web browser using any conventional techniques. Web code can issue an API call to the web browser that requests display parameters for the web browser and/or XR system 404, such as a request to determine whether the web browser and XR system are capable of rendering 3D content. In some implementations, the web code can be: a) client-side code provided by web source 408 running at the web browser; or b) server-side code running at web source 408 or any other suitable computing device.

The web browser can respond to the API call with a binary value (e.g., 0 or 1) that indicates whether the web browser and XR system are capable of rendering 3D content. Once the web code receives a response that indicates 3D display capabilities, the web code can issue a second API call that signals to the web browser that 3D version(s) of the webpage will be provided. For example, the second API call can generate an asynchronous promise that is fulfilled once the 3D version(s) are provided/loaded. The web code can generate stereoscopic versions of the webpage for XR system 404/the web browser, and the stereoscopic versions can be displayed by the web browser in a 3D mode (e.g., via an HMD of XR system 404).

In some implementations, the web browser can also expose an API to the web code that provides the current display mode for the web browser. For example, the current display mode of 2D mode or 3D mode can be provided by the web browser to the web code via the API call.

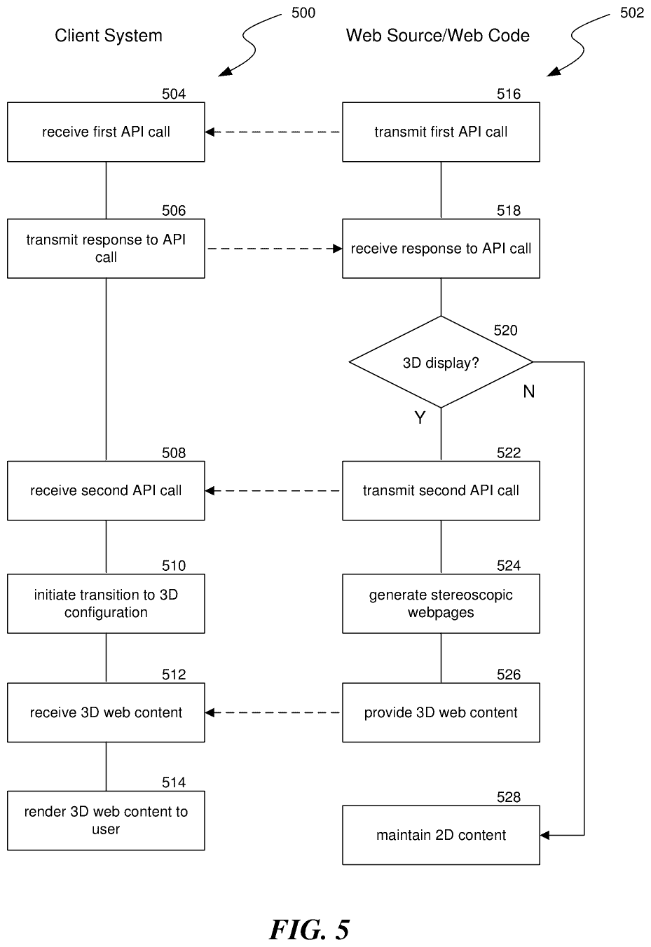

FIG. 5 is a flow diagram illustrating a process used in some implementations of the present technology for dynamically displaying three-dimensional web content to a user. In some implementations, processes 500 and 502 can be performed when an XR browser loads a webpage or when the XR browser requests a webpage that includes 3D configurable content. In some implementations, process 500 can be performed by a client system that runs the XR browser and process 502 can be performed by web code running at a web source (e.g., content delivery network, web host, server, etc.) or web coded running at the XR browser loaded by the web source.

In some implementations, processes 500 and 502 can be performed after a 2D version of a webpage is provided to the XR browser. In another example, processes 500 and 502 can be performed upon request for a webpage and prior to any version of the webpage being provided to the XR browser.

At block 504, process 500 can receive a first API call. For example, an XR browser implemented by the client system can receive an API call from the web code that implements a webpage. The XR browser can expose an API to the web code that permits coordination between the XR browser and web code. The API and API call can be implemented by any suitable technique that permits communication between web code and a web browser.

At block 506, process 500 can transmit a response to the first API call. For example, the first API call can be a request for display parameters, such as a request for a binary answer to whether or not the XR browser and client system that implements the XR browser are capable of 3D display. At block 508, process 500 can receive a second API call. For example, when the web code receives a positive reply to the first API call (e.g., indicating that the XR browser and client system are capable of 3D display) the web code can issue the second API call.

In some implementations, the second API call indicates to the XR browser that the web code/web source will provide 3D version(s) of the webpage, such as stereoscopic versions. For example, the second API call can create an asynchronous promise that is fulfilled when the 3D version(s) are provided to the web browser and/or resource for the 3D version(s) are loaded.

At block 510, process 500 can initiate a transition to a 3D configuration for the XR browser. For example, the second API call signals that 3D content will be provided to the XR browser. Accordingly, the transition from a 2D mode for the XR browser to a 3D mode can be initiated. At block 512, process 500 can receive 3D version(s) of the webpage from the web code. For example, stereoscopic versions of the webpage can be generated by the web code. In some implementations, other resources (e.g., images, videos, 3D models, etc.) may be loaded at the web browser to support display of the 3D version(s) of the webpage.

At block 514, process 500 can render the 3D version(s) of the webpage to the user via the XR browser and the XR system. For example, the XR browser and XR system can transition to a 3D mode such that stereoscopic versions of the webpage are displayed to the user in a manner that creates a 3D effect for at least portions of content of the webpage. The portions of content rendered in 3D can include images, videos, text, interface components (e.g., buttons, panels, etc.), and any other suitable web content.

In some implementations, process 502 can be performed by web code at the XR browser and/or server-side code at the web source. At block 518, process 502 can transmit the first API call. For example, the XR browser can expose an API to the web code that permits coordination between the XR browser and web code. In some implementations, the web code can issue an API call to the exposed API. At block 518, process 502 can receive a response to the first API call. For example, the web browser can issue a response to the API call that is received by the web code.

At block 520, process 502 can determine whether the XR browser and/or XR system are capable of 3D display. For example, the first API call can be a request for display parameters, such as a request for a binary answer to whether or not the XR browser and XR system that implements the XR browser are capable of 3D display. Based on the API call, process 502 can determine whether or not the XR browser and XR system are capable of 3D display. When the XR browser and/or XR system are capable of 3D display, process 502 can progress to block 522. When the XR browser and/or XR system are not capable of 3D display, process 502 can progress to block 528.

At block 522, process 502 can transmit a second API call to the XR browser. For example, the second API call can indicate to the XR browser that the web code will provide 3D version(s) of the webpage, such as stereoscopic versions. At block 524, process 502 can generate stereoscopic versions of the webpage. For example, web code can generate two stereoscopic versions of the webpage (e.g., left eye and right eye, top and bottom, etc.) such that at least portions of the content displayed in the stereoscopic versions, when viewed by a user via an XR system, are perceived by the user as three-dimensional.

At block 526, process 500 can provide the 3D webpage version(s) to the XR browser. For example, the web code can generate the stereoscopic versions of the webpage and provide the versions to the XR browser. In some implementations, additional resources (e.g., images, videos, 3D models, etc.) can be loaded at the XR browser that support the 3D webpage rendering.

At block 528, process 502 can maintain a 2D display of the webpage. For example, when the second API call returns a ‘False’ or negative, the reply indicates that the XR browser and/or XR system are not capable of 3D displays, and thus the 2D version of the webpage is maintained (e.g., provided or continued to be provided).

Implementations generate stereoscopic versions of the webpage using any suitable techniques. For example, logic present in the webpage/web code can be triggered to generate stereoscopic versions of the webpage (e.g., when the response to the first API call indicates that the browser/XR system are capable of 3D display). In some implementations, the 3D mode for a XR browser can split a viewport (e.g., horizontally, vertically, etc.) and the stereoscopic versions of the webpage can be displayed at each side of the split viewport. In an example, the web code can use canvas element(s) (e.g., an HTML5 canvas element) to selectively draw stereoscopic content in the stereoscopic versions of the webpage. In another example, the web code can trigger the placement of stereoscopic versions of a full screen video.

In some implementations, tags or styles (e.g., CSS class tags, CSS styles, etc.) can be used to selectively position and configure content in the stereoscopic versions of the webpage such that the content is perceived with depth when a user views the content via a XR system. For example, JavaScript logic and/or CSS tags can be used to duplicate a webpage into two versions and reposition content items within the two versions to support stereoscopic display of the two versions via an XR system. In some implementations, the web code logic can trigger content item substitutions such that stereoscopic versions of content items (e.g., images, videos, etc.) are substituted in the stereoscopic versions of the webpages.

In another example, DOM trees can represent two stereoscopic versions of the webpage, the versions positioning and configuring one or more content items such that, when a user views the content items via an XR system, the content is perceived with depth (e.g., in 3D). For example, classes at the root of both DOM trees can correspond to specific stereoscopic versions (e.g., left eye and right eye) and transformations can select these classes to position and configure the content. In another example, an HTML header tag (or any other suitable HTML tag) can define the two stereoscopic versions of the webpage so that content, when viewed by a user via an XR system, is perceived with depth. For example, web code (e.g., JavaScript, PHP, etc.) can trigger the generation of HTML tag(s) that position and configure the webpage content.

In some implementations, the entirety of a webpage is positioned and configured so that the webpage, when viewed by a user via an XR system, is perceived with depth in the stereoscopic versions. In other implementations, select content is positioned and configured such that the content is perceived with depth when viewed by a user via an XR system in the stereoscopic versions. For example, content comprising a predefined tag, attribute, and/or property can cause the content to be selectively positioned and configured to cause a 3D effect when viewed by a user via an XR system.

In some implementations, the content provided by the source can be dynamic itself such that, when the XR system displays the web browser in a 2D mode, a 2D version of the web page is displayed and, when the XR system displays the web browser in a 3D mode, a 3D version of the web page is displayed. For example, web code can be configured to detect display parameters, such as a display mode for the XR browser, display hardware (e.g., two coordinated display devices), a split viewport, etc. In some implementations, logic in the web code (e.g., JavaScript, PHP: Hypertext Preprocessor (PHP), CSS media query, etc.) can detect when the hardware and/or XR browser are in a state to display stereoscopic versions of a webpage (e.g., are in 3D mode). For example, a CSS media query can trigger stereoscopic versions of the webpage when the query returns that the webpage is being displayed in a 3D capable manner (e.g., a head-mounted display of an XR system). In another example, the XR system can expose an API that can return (to the web code) the display state for the device (e.g., whether the XR system is in 3D mode).

The web code can trigger generation of the stereoscopic versions of the webpage (for rendering) based on this detection. Any suitable technique can be used to generate the stereoscopic versions of the webpage upon detection that the client system and/or XR browser are in a state to display 3D content. When the logic in the web code detects that the client system and/or XR browser are not in a state to display 3D content (e.g., not in 3D mode) the logic can generate a traditional 2D webpage for display at the XR browser/XR system.

In some implementations, when the XR browser is transitioned to a 3D mode by web code (e.g., based on an API call that indicates stereoscopic versions of a web page will be rendered), interface elements of the XR browser itself can also be rendered in 3D. For example, transitioning the XR browser to 3D mode can cause menu(s), menu item(s), button(s), and other suitable elements of a browser chrome to render in 3D (e.g., render with a positioning and configuration such that a user perceives the elements of the browser with depth).

The technology is directed to decentralizing an entitlement by issuing it as a token embedded with metadata specifying its features. The metadata can include information such as who the token was issued to, who the token was issued by (e.g., as a link to an issuing authority's public certificate), when the token was issued, when the token is valid, claims representing the entitlement, etc. The token can further include a signature block of the issuing authority encoded with a private key. Because the token provides all of the data needed to load the entitlement without the issuing authority, a different authority can validate and grant the entitlement by decoding the signature block with a public key associated with the issuing authority to confirm its authenticity.

Some implementations can issue the token to a messaging account associated with a recipient user, such as an e-mail or text message account identified in the metadata of the token, making the token unique to the recipient user. Implementations can store the token in an e-mail or text message system instead of using a digital wallet, allowing an existing system to provide easy entitlement management, which in some cases can be automated, e.g., through tags embedded in the e-mail or text message specifying it as containing an entitlement token. The token can be represented by a readable text block, an encoded text block, and/or a Quick Response (QR) code.

As used herein, an “entitlement” can be a right to use, access, or consume a resource, such as an application (e.g., via a temporary or permanent license), a service, an item (e.g., a virtual object, a virtual world, an avatar, etc.), an account (e.g., a subscription to a particular creator), and the like. An entitlement can also be referred to as an authorization, access right, permission, rule, and/or privilege with respect to a resource.

As used herein, a “private key” and “public key” can be a pair of linked cryptographic keys generated by an authority (e.g., a server associated with an issuing entity). A private key can be used to private functions, such as applying a digital signature on a token. The private key can be kept in a secure storage medium and can typically not be known by other authorities (e.g., a server associated with a validating authority). The public key can be used for public functions, such as for verifying a digital signature on a token supposedly issued by an authority (e.g., a server associated with an issuing entity).

The implementations described herein provide improvements to the technical field of entitlements in that they are not tied to one specific platform and are thus freely portable. Some implementations can use an existing e-mail account as a “wallet” for a token associated with an entitlement, without requiring users to create a new account on a separate application to store the token. Some implementations allow an authority other than an issuing authority to verify that the user is the person the entitlement was issued to, eliminating sharing of entitlements between users. Further, implementations eliminate the need to store the token on a blockchain wallet, which can require a separate log-in that can be lost or hacked.

For example, a user can be a fan of a creator “Doofus” and sign up for a VIP subscription to “Doofus” on an issuing application. The user can get a token that he can submit to the “Doofus” server on other applications to prove his VIP status. The other applications advantageously do not need to make calls to the issuing application to validate the VIP status. Further, even if the issuing application were to suspend the user's account, his entitlement to the VIP subscription would still be valid elsewhere.

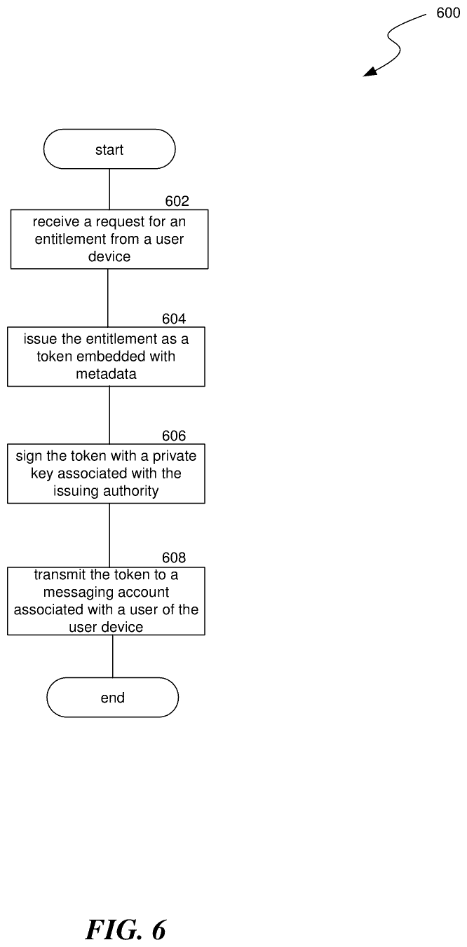

FIG. 6 is a flow diagram illustrating a process 600 used in some implementations for issuing decentralized entitlements by an issuing authority. In some implementations, process 600 can be performed as a response to a user request for an entitlement. In some implementations, process 600 can be performed as a response to a user request for a token associated with an issued entitlement. In some implementations, process 600 can be performed by an issuing authority, i.e., a server that can issue the entitlement and/or the token associated with an issued entitlement.

At block 602, process 600 can receive a request for an entitlement. The entitlement can be a right with respect to a resource, such as an application (e.g., via a temporary or permanent license), a service, an item (e.g., a virtual object, a virtual world, an avatar, etc.), an account (e.g., a subscription to a particular creator), and/or the like.

At block 604, process 600 can issue the entitlement as a token embedded with metadata specifying features of the entitlement. The metadata can include, for example, who the token was issued to, who the token was issued by, a Global Unique Identification Number (GUID), an issued time, a valid start time and date, a valid end time and date, and claims that represent the entitlement, as described further herein. When the entitlement is a subscription to a particular creator, the claims can include, for example a type of subscription, an identifier associated with the creator, and/or a level of subscription. When the entitlement is to a virtual item, the claims can include, for example, an item type, an item bonus, and/or an item quantity.

At block 606, process 600 can sign the token with a private key associated with the issuing authority. The private key can be kept in a secure storage medium associated with the issuing authority and not known by other authorities (e.g., a validating authority). Process 600 can create and append the signature to the metadata specifying the features of the entitlement. For example, the signature can be a base64 encoded block.

At block 608, process 600 can transmit the token to a messaging account associated with a user of the user device, thereby binding the token to the messaging account. The user device can access the token via the messaging account, i.e., the messaging account becomes a “wallet” for the token. Thus, the token cannot be stolen by other parties or resold to other parties not associated with that messaging account even if it is forwarded to other users.

In some implementations, process 600 can validate the messaging account associated with the user prior to transmitting the token to the messaging account, such as, for example, sending a message with a hyperlink that, when selected, redirects to a website associated with the issuing authority that proves that the messaging account is valid. The user device can further transmit the token to a validating authority different than the issuing authority. The validating authority can authenticate the token with a public key associated with the issuing authority. The validating authority can further load the entitlement using the metadata specifying the features of the entitlement, as described further herein.

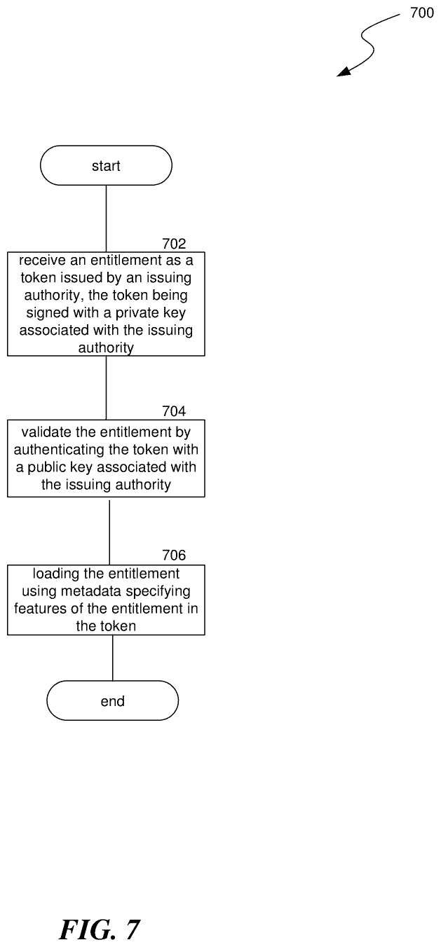

FIG. 7 is a flow diagram illustrating a process 700 used in some implementations for granting decentralized entitlements by a validating authority. In some implementations, process 700 can be performed as a response to receiving an entitlement as a token from a user device. In some implementations, process 700 can be performed by a validating authority, i.e., a server that can validate and/or load the entitlement and/or the token associated with an issued entitlement. The validating authority can be different than the issuing authority.

At block 702, process 700 can receive an entitlement as a token issued by an issuing authority. For example, a user can access her messaging account, search for the token, then copy and paste the token into a form generated by the validating authority. The token can be signed with a private key associated with the issuing authority that is hidden from the validating authority. The token can include metadata specifying features of the entitlement. The metadata can include, for example, who the token was issued to, who the token was issued by, a Global Unique Identification Number (GUID), an issued time, a valid start time and date, a valid end time and date, and claims that represent the entitlement, as described further herein.

At block 704, process 700 can validate the entitlement by authenticating the token with a public key associated with the issuing authority. Process 700 can authenticate the token by validating the signature on the token that was encoded with the private key known by the issuing authority. Because the private key is not known by the validating authority, the validating authority can decode the signature by using a public key associated with the issuing authority that is known to the validating authority.

At block 706, process 700 can load the entitlement using the metadata specifying the features of the entitlement in the token. For example, process 700 can identify the entitlement using the claims specified in the metadata. The claims of the entitlement can be to a resource, such as an application (e.g., via a temporary or permanent license), a service, an item (e.g., a virtual object, a virtual world, an avatar, etc.), an account (e.g., a subscription to a particular creator), and/or the like. When the entitlement is a subscription to a particular creator, the claims can include, for example a type of subscription, an identifier associated with the creator, and/or a level of subscription. When the entitlement is to a virtual item, the claims can include, for example, an item type, an item bonus, and/or an item quantity. The metadata can further specify when process 700 can load the entitlement, i.e., a valid start and end date and time in which the entitlement is valid.

In some implementations, process 700 can be performed automatically when the message including the token is opened in the messaging account. For example, the message (e.g., an e-mail or text message) can include embedded tags specifying that it includes an entitlement. Thus, the messaging account can automatically identify that the message includes an entitlement and initiate process 700.

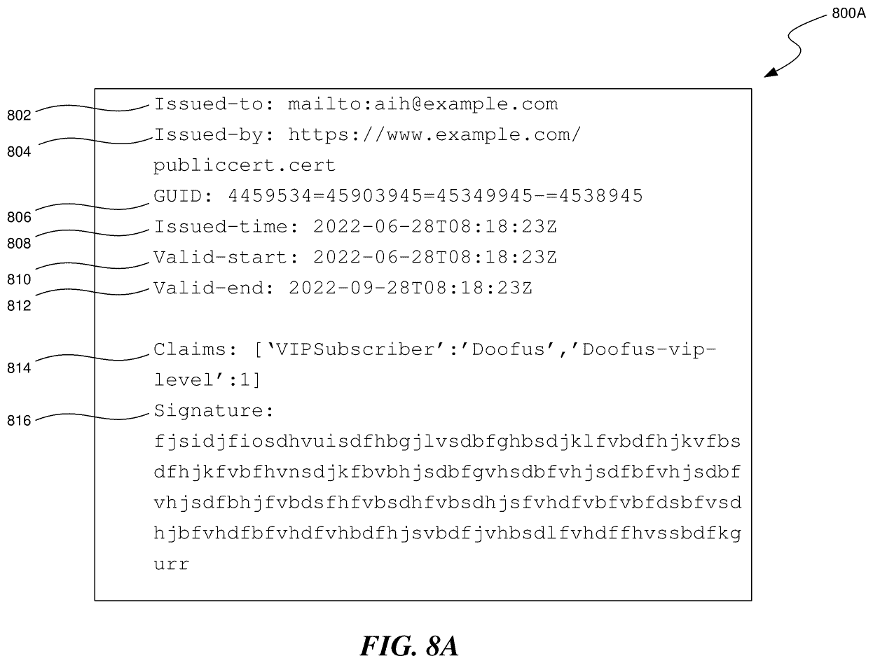

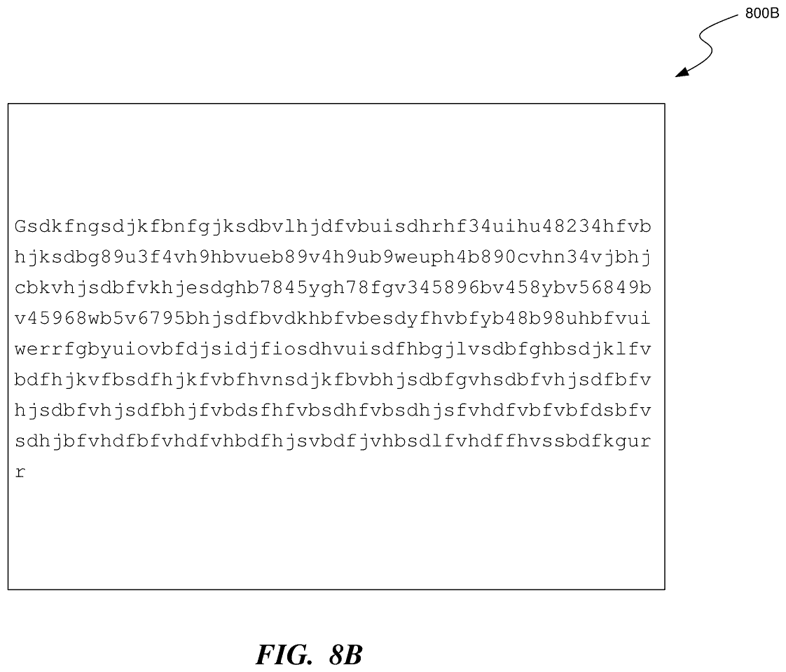

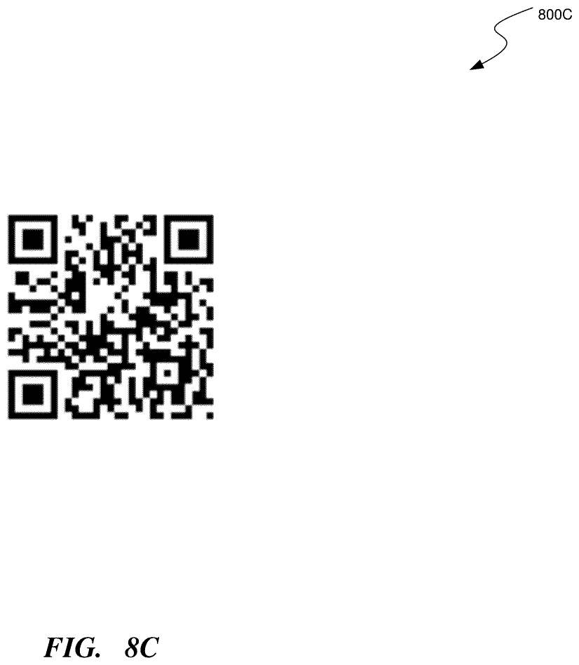

FIG. 8A is a conceptual diagram illustrating an example token 800A as a readable text block in accordance with some implementations of the present technology. Token 800A can represent a user entitlement. In some implementations, token 800A can be provided to a validating authority who can validate and/or load the entitlement represented by token 800A. Token 800A can include a plurality of metadata (i.e., issued-to 802, issued-by 804, GUID 806, issued-time 808, valid-start 810, valid-end 812, claims 814) and signature 816.

Issued-to 802 can be the messaging address to which token 800A was issued. For example, issued-to 802 can be represented by a uniform resource identifier (URI), e-mail address, or phone number. In this example, issued-to 802 is “mailto:aig@example.com.”

Issued-by 804 can be an identifier of the issuing authority who issued token 800A. For example, issued-by 804 can be a URI to the issuing authority's public certificate. In this example, issued-by 804 is “https://www.example.com/publiccert.cert.” In some implementations, a validating authority can decide which certificates it will treat as valid, such as certificates issued by a particular issuing authority or a group of issuing authorities.

GUID 806 can be a globally unique identifier representing the entitlement associated with token 800A. In this example, GUID 806 can be “4459534=45903945=45349945−=4538945.”

Issued-time 808 can be the date and time in which token 800A was issued. In this example, issued-time 808 is “2022-06-28T08:18:23Z.” Valid-start 810 can be the starting date and time for which token 800A (and its underlying entitlement) is valid. In this example, valid-start 810 is “2022-06-28T08:18:23Z.” Valid-end 812 can be the end date and time for which token 800A (and its underlying entitlement) is valid. In this example, valid-end 812 is “2022-09-28T08:18:23Z.” Through valid-start 810 and valid-end 812, an entitlement can have a specific duration after which it is no longer valid. In some implementations, token 800A can omit valid-end 812, i.e., token 800A (and its underlying entitlement) is valid indefinitely.

Claims 814 can be an arbitrary set of claims that represent the entitlement. For example, claims 814 can include the name of a creator (e.g., “Doofus”), a subscription type (e.g., “VIPSubscriber”), and a subscription level (e.g., “Doofus-vip-level:1”). In another example in which the entitlement is a virtual object, claims 814 can include an item type (e.g., sword), an item bonus (e.g., +3), and an item quantity (e.g., 1).

In some implementations, issued-to 802, issued-by 804, guid 806, issued-time 808, valid-start 810, valid-end 812, and claims 814 can be encoded in JavaScript Object Notation (JSON). Signature 816 can be created and appended to the JSON block, e.g., as a base64 encoded block.

FIG. 8B is a conceptual diagram illustrating an example encoded token 800B in accordance with some implementations of the present technology. Token 800B can represent a user entitlement. In some implementations, token 800B can be a base64 (or other encoding) of token 800A. In this example, token 800B is “GsdkfngsdjkfbnfgjksdbvIhjdfvbuisdhrhf34uihu48234hfvbhjksdbg89u3f4vh9hbvueb89v4h9 ub9weuph4b890cvhn34vjbhjcbkvhjsdbfvkhjesdghb7845ygh78fgv345896bv458ybv56849bv 45968wb5v6795bhjsdfbvdkhbfvbesdyfhvbfyb48b98uhbfvuiwerrfgbyuiovbfdjsidjfiosdhvuisdf hbgjlvsdbfghbsdjklfvbdfhjkvfbsdfhjkfvbfhvnsdjkfbvbhjsdbfgvhsdbfvhjsdfbfvhjsdbfvhjsdfbhjfv bdsfhfvbsdhfvbsdhjsfvhdfvbfvbfdsbfvsdhjbfvhdfbfvhdfvhbdfhjsvbdfjvhbsdIfvhdffhvssbdfkgur r.” In some implementations, token 800B can be provided to a validating authority who can validate and/or load the entitlement represented by token 800B, as described further herein.

FIG. 8C is a conceptual diagram illustrating an example Quick Response (QR) code 800C representing a token in accordance with some implementations of the present technology. QR code 800C can represent a token associated with a user entitlement. In some implementations, QR code 800C can represent token 800A and/or token 800B. In some implementations, QR code 800C can be scanned or otherwise provided to a validating authority who can validate and/or load the entitlement represented by the token.

An energy expenditure calculation system can assess a total caloric expenditure of a user while operating one or more devices for an artificial reality (XR) system. In particular, the energy expenditure calculation system can automatically assess, for lower body movements which can be directly unaccounted for by system tracking components, caloric amounts attributable to those movements. For instance, the system can, for pose data tracked for the user, employ a kinematic model to infer such movements. In this regard, the kinematic model can define, according to anatomical capabilities and constraints, a current body configuration of the user that can yield one or more lower body postures of the user. In view of the observed postures, the system can then employ a machine learning model to evaluate, for physiological attributes associated with the user for one or more changes in the postures, an ensuing caloric expenditure. This expenditure can then be combined with correlated caloric expenditures evaluated for any upper body movement(s) of the user while operating the one or more XR devices to reach a total energy expenditure.

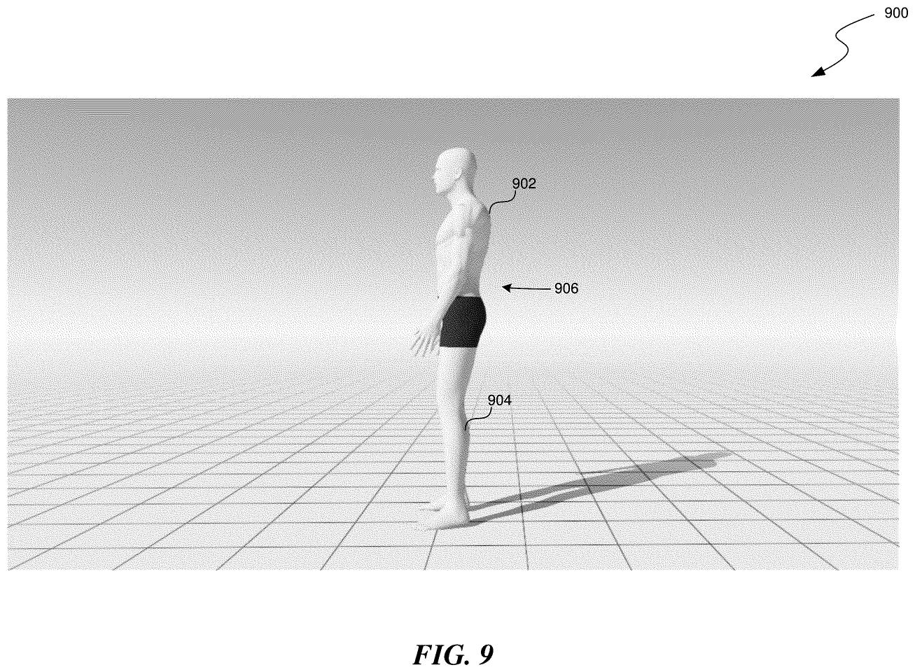

FIG. 9 is a conceptual diagram 900 illustrating an XR system user 902 disposed in a first pose. For instance, such first pose can be defined according to a standing position in which the user is in an upright posture, where the user's legs 904 are substantially parallel to the torso 906 position. As can be understood, the user can be oriented in the first pose while preparing to engage in activities for an associated artificial reality system.

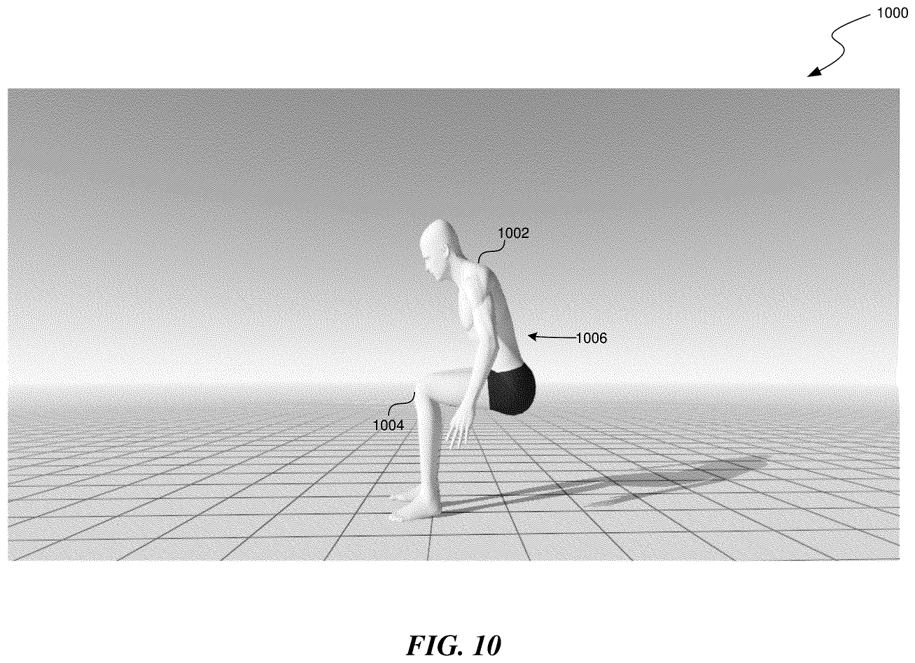

FIG. 10 is a conceptual diagram 1000 illustrating an XR system user 1002 disposed in a second pose. As can be understood, such second pose can, for an XR system with which the user is engaged, represent an initial posture for the user or a transitioned posture, i.e., a posture displacing the user's torso 1006 and legs 1004 relative to each other. Some previous XR systems are unable to account for the difference in user energy expenditure due to the change from the first pose, discussed above, to the second pose.

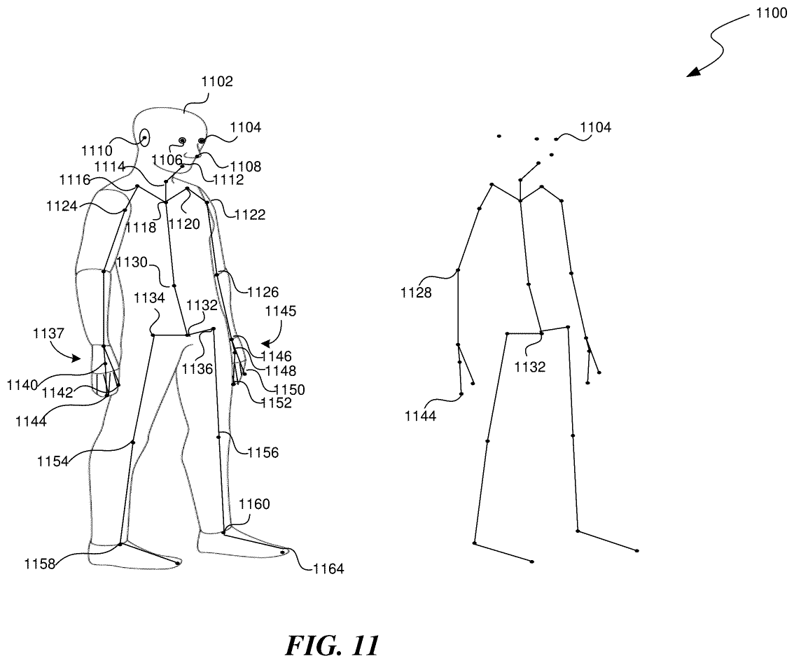

FIG. 11 is a conceptual diagram 1100 illustrating an example kinematic model of a user for an XR system. On the left side, example 1100 illustrates points defined on a body of a user 1102 while these points are again shown on the right side of FIG. 11 without the corresponding person to illustrate the actual components of the kinematic model. These points include eyes 1104 and 1106, nose 1108, ears 1110 (second ear point not shown), chin 1112, neck 1114, clavicles 1116 and 1120, sternum 1118, shoulders 1122 and 1124, elbows 1126 and 1128, stomach 1130, pelvis 1132, hips 1134 and 1136, hands 1137 and 1145, wrists 1138 and 1146, palms 1140 and 1148, thumb tips 1142 and 1150, finger tips 1144 and 1152, knees 1154 and 1156, ankles 1158 and 1160, and tips of feet 1162 and 1164. In various implementations, more or less points are used in the kinematic model. Some corresponding labels have been put on the points on the right side of FIG. 11, but some have been omitted to maintain clarity. Points connected by lines show that the kinematic model maintains measurements of distances and angles between certain points. Because points 1104-1110 are generally fixed relative to point 1112, they do not need additional connections.

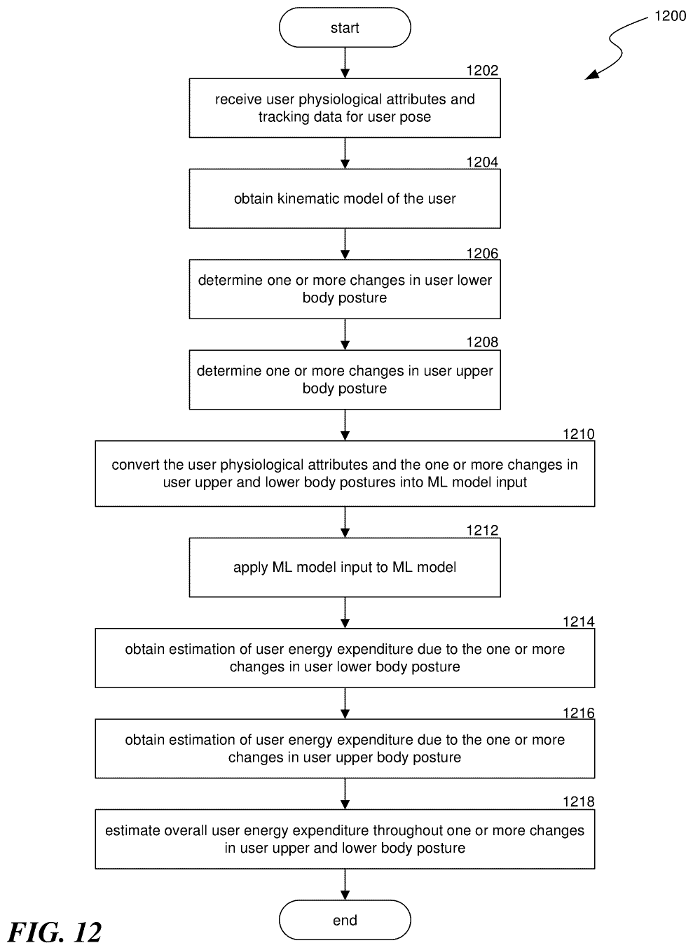

FIG. 12 is a flow diagram illustrating a process 1200 used in some implementations for determining a total energy expenditure of a user due to movements for an XR environment. For instance, the total energy expenditure can be associated with upper and/or lower body movements of the user. In some implementations, process 1200 can be initiated by a user executing a program to create an XR environment. In some implementations, one or more portions of process 1200 can be performed on a server system in control of that XR environment; while in some cases all or parts of process 1200 can be performed on an XR client device.

At block 1202, process 1200 can receive one or more physiological attributes of the user for the XR environment while the user interacts with the corresponding XR system. Exemplary attributes can include, for instance, the user's height, weight, age, heart rate, pulse rate, oxygen saturation, etc. Additionally, process 1200 can receive, during one or more interactions for the XR environment, tracking data for the user's pose resulting from her upper and/or lower body movements. In some implementations, the tracking data can be sourced from the user's headset or sensors in her hand controllers while interacting with the XR environment. A non-exhaustive list of elements that can define the pose can include a positioning of one or more of the user's head, a hand or hands of the user, an arm or arms of the user, and a leg or legs of the user.

At block 1204, process 1200 can, using the tracking data received at block 1202, obtain a kinematic model of the user, as has been discussed above. For instance, process 1200 can map the tracking data to the kinematic model in order to define a pose of the user according to relative positioning for the user's head, hands, arms, legs, etc.

At block 1206, process 1200 can, using the kinematic model obtained at block 1204, determine one or more changes in lower body posture of the user throughout the collection of tracking data received at block 1202. In other words, process 1200 can continually monitor changes in the user's lower body movements that may define corresponding changes in the user's lower body posture when interacting within an XR environment. For example, such changes in lower body posture can be the result of repositioning of the user's legs. Alternatively or in addition, it is contemplated that changes in lower body posture of a user can encompass movements of one or more portions of the user's abdominal and/or back muscles. In some implementations, process 1200 can determine the changes in lower body posture relative to temporal changes in elevation of the user's head above a known baseline (e.g., a ground surface or an object of a certain known height), as indicated by a headset worn by the user while interacting within the XR environment. In other implementations, process 1200 can determine the changes in lower body posture of the user according to one or more reductions in relative standing height of the user while wearing the headset.

At block 1208, process 1200 can, also using the kinematic model obtained at block 1204, determine one or more changes in upper body posture of the user where such changes correspond to the tracking data received at block 1202. In exemplary instances, the changes can represent different elevations and/or lateral orientations for the user's head, arms, chest area, etc., as the user moves between a variety of positions that can be induced by interaction with an XR environment.

At block 1210, process 1200 can convert the user's physiological attributes and the one or more changes in user upper and/or lower body posture into machine learning model input. For example, one or more of the physiological attributes, e.g., height, weight, and age, can be accorded certain data formats—such as values entered in corresponding vector slots. The kinematic model can be encoded into values representing the body posture (i.e., upper and/or lower body posture). These inputs are translated into numerical data that the machine learning model has been trained to receive.

At block 1212, process 1200 can apply the machine learning model input to a machine learning model. A “machine learning model” or “model” as used herein, refers to a construct that is trained using training data to make predictions or provide probabilities for new data items, whether or not the new data items were included in the training data. For example, training data for supervised learning can include positive and negative items with various parameters and an assigned classification. Examples of models include: neural networks (traditional, deep, convolution neural network (CSS), recurrent neural network (RNN)), support vector machines, decision trees, decision tree forests, Parzen windows, Bayes, clustering, reinforcement learning, probability distributions, decision trees, and others. Models can be configured for various situations, data types, sources, and output formats.

The machine learning model can be trained with supervised learning and use training data that can be obtained from physiological attributes of prior XR system users while those users interact with XR systems. Exemplary physiological attributes can include height, weight, age, heart rate, pulse rate, and oxygen saturation. More specifically, each item of the training data can, for a change in user upper and/or lower body posture, include an instance of a user's height, weight, and/or age matched to an indication of breathing intensity (e.g., a user's heart rate, pulse rate, and/or oxygen saturation), which may have been gathered for training data while the user was being monitored by specialized energy consumption monitoring equipment, such as a respirometer that measures a difference between carbon dioxide breathed out and oxygen breathed in. The matching can be performed according to known relationships for the physiological attributes during one or more periods of physical exertion while transitioning between postures. For instance, the matching can be a result of different types of physical exertion causing one or more patterns in heart rate, pulse rate, and/or oxygen saturation data. During the model training a representation of the physiological attributes (e.g., values representing the attributes for respective transitions to upper and/or lower body postures, etc.) can be provided to the model. Then, the output from the model, one or more estimations of energy (i.e., caloric) expenditure due to physical exertion associated with a change in upper and/or lower body posture, can be compared to an actual amount of energy expenditure for the physical exertion and, based on the comparison, the model can be modified, such as by changing weights between nodes of the neural network or parameters of the functions used at each node in the neural network (e.g., applying a loss function). After applying each of the pairings of the inputs (physiological attributes of a user for changes in upper and/or lower body postures) and the desired output (estimated energy expenditure due to a change in user upper and/or lower body posture) in the training data and modifying the model in this manner, the model is trained to evaluate new instances of energy expenditure arising from a user changing her posture while interacting with an XR system.

At block 1214, process 1200 can obtain an estimation of a user's energy expenditure due to the one or more changes in lower body posture determined at block 1206. For instance, process 1200 can assign, for each respective change in lower body posture mapped for physiological attributes (height, weight, etc.), an estimated number (i.e., amount) of calories expended by the user to transition from one posture to another. As an example, process 1200 can, using the physiological attributes of the user and a detected change in elevation of the user's head with respect to a baseline (i.e., ground surface), assign a caloric expenditure due to that change. That is, process 1200 can, for a change in lower body posture of the user from a posture in which she is standing to one in which she is squatting, assign a corresponding number of calories estimated to achieve the changed posture.

At block 1216, process 1200 can obtain an estimation of a user's energy expenditure due to the one or more changes in upper body posture determined at block 1208. For example, process 1200 can, for changes in one or more of the user's lateral head positioning and vertical arm positioning of her arms and/or chest mapped for physiological attributes, assign an estimated amount of calories expended when obtaining the changed position(s).

At block 1218, process 1200 can estimate an overall energy expenditure throughout one or more changes in user upper and lower body posture. For instance, process 1200 can variously combine energy expenditures attributable to upper and lower body movements of a user when making the overall energy expenditure estimation. That is, process 1200 can detect (e.g., via an inertial measurement unit (IMU)) a whole-body change in pose due to upper and lower body movements such as where there is a detected change in elevation from a baseline to a user's headset combined with a detected change in lateral movement of the headset. In view of energy expenditures assigned for such detected movements, process 1200 can account for a total energy expenditure of a user while the user engages in an XR environment. This way, the user can be apprised of, for instance, an exercise value that can be associated with undertaking activities for the XR environment.

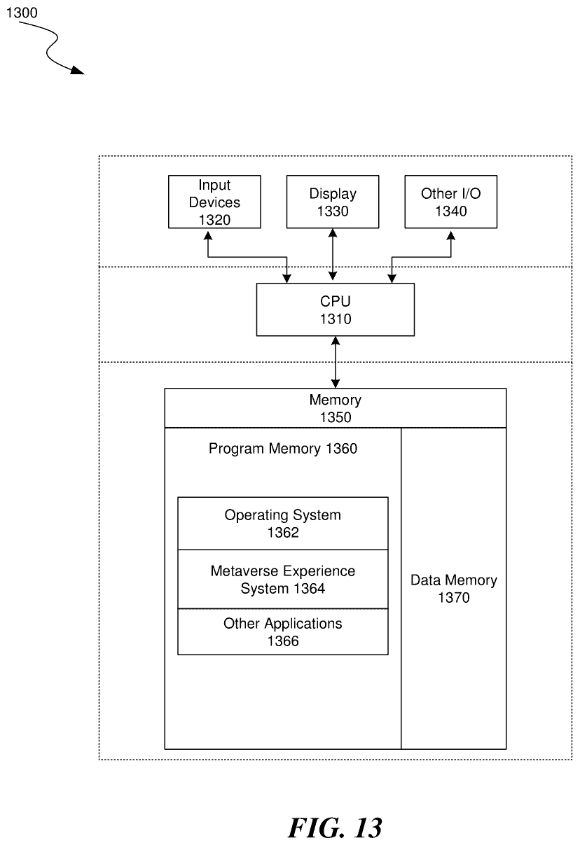

FIG. 13 is a block diagram illustrating an overview of devices on which some implementations of the disclosed technology can operate. The devices can comprise hardware components of a device 1300 as shown and described herein. Device 1300 can include one or more input devices 1320 that provide input to the Processor(s) 1310 (e.g., CPU(s), GPU(s), HPU(s), etc.), notifying it of actions. The actions can be mediated by a hardware controller that interprets the signals received from the input device and communicates the information to the processors 1310 using a communication protocol. Input devices 1320 include, for example, a mouse, a keyboard, a touchscreen, an infrared sensor, a touchpad, a wearable input device, a camera- or image-based input device, a microphone, or other user input devices.

Processors 1310 can be a single processing unit or multiple processing units in a device or distributed across multiple devices. Processors 1310 can be coupled to other hardware devices, for example, with the use of a bus, such as a PCI bus or SCSI bus. The processors 1310 can communicate with a hardware controller for devices, such as for a display 1330. Display 1330 can be used to display text and graphics. In some implementations, display 1330 provides graphical and textual visual feedback to a user. In some implementations, display 1330 includes the input device as part of the display, such as when the input device is a touchscreen or is equipped with an eye direction monitoring system. In some implementations, the display is separate from the input device. Examples of display devices are: an LCD display screen, an LED display screen, a projected, holographic, or augmented reality display (such as a heads-up display device or a head-mounted device), and so on. Other I/O devices 1340 can also be coupled to the processor, such as a network card, video card, audio card, USB, firewire or other external device, camera, printer, speakers, CD-ROM drive, DVD drive, disk drive, or Blu-Ray device.

In some implementations, the device 1300 also includes a communication device capable of communicating wirelessly or wire-based with a network node. The communication device can communicate with another device or a server through a network using, for example, TCP/IP protocols. Device 1300 can utilize the communication device to distribute operations across multiple network devices.

The processors 1310 can have access to a memory 1350 in a device or distributed across multiple devices. A memory includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, and so forth. A memory is not a propagating signal divorced from underlying hardware; a memory is thus non-transitory. Memory 1350 can include program memory 1360 that stores programs and software, such as an operating system 1362, metaverse experience system 1364, and other application programs 1366. Memory 1350 can also include data memory 1370, which can be provided to the program memory 1360 or any element of the device 1300.

Some implementations can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, gaming consoles, tablet devices, multiprocessor systems, microprocessor-based systems, set-top boxes, programmable consumer electronics, network PCs, minicomputers, mainframe computers, distributed computing environments that include any of the above systems or devices, or the like.

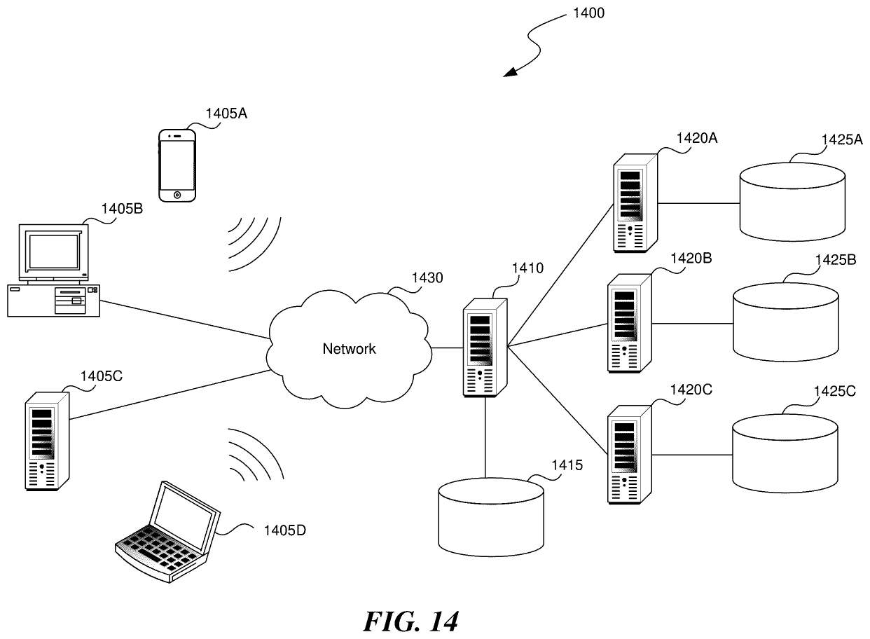

FIG. 14 is a block diagram illustrating an overview of an environment 1400 in which some implementations of the disclosed technology can operate. Environment 1400 can include one or more client computing devices 1405A-D, examples of which can include device 1300. Client computing devices 1405 can operate in a networked environment using logical connections through network 1430 to one or more remote computers, such as a server computing device.

In some implementations, server 1410 can be an edge server which receives client requests and coordinates fulfillment of those requests through other servers, such as servers 1420A-C. Server computing devices 1410 and 1420 can comprise computing systems, such as device 1300. Though each server computing device 1410 and 1420 is displayed logically as a single server, server computing devices can each be a distributed computing environment encompassing multiple computing devices located at the same or at geographically disparate physical locations. In some implementations, each server 1420 corresponds to a group of servers.

Client computing devices 1405 and server computing devices 1410 and 1420 can each act as a server or client to other server/client devices. Server 1410 can connect to a database 1415. Servers 1420A-C can each connect to a corresponding database 1425A-C. As discussed above, each server 1420 can correspond to a group of servers, and each of these servers can share a database or can have their own database. Databases 1415 and 1425 can warehouse (e.g., store) information. Though databases 1415 and 1425 are displayed logically as single units, databases 1415 and 1425 can each be a distributed computing environment encompassing multiple computing devices, can be located within their corresponding server, or can be located at the same or at geographically disparate physical locations.

Network 1430 can be a local area network (LAN) or a wide area network (WAN), but can also be other wired or wireless networks. Network 1430 may be the Internet or some other public or private network. Client computing devices 1405 can be connected to network 1430 through a network interface, such as by wired or wireless communication. While the connections between server 1410 and servers 1420 are shown as separate connections, these connections can be any kind of local, wide area, wired, or wireless network, including network 1430 or a separate public or private network.

Embodiments of the disclosed technology may include or be implemented in conjunction with an artificial reality system. Artificial reality or extra reality (XR) is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured content (e.g., real-world photographs). The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may be associated with applications, products, accessories, services, or some combination thereof, that are, e.g., used to create content in an artificial reality and/or used in (e.g., perform activities in) an artificial reality. The artificial reality system that provides the artificial reality content may be implemented on various platforms, including a head-mounted display (HMD) connected to a host computer system, a standalone HMD, a mobile device or computing system, a “cave” environment or other projection system, or any other hardware platform capable of providing artificial reality content to one or more viewers.

“Virtual reality” or “VR,” as used herein, refers to an immersive experience where a user's visual input is controlled by a computing system. “Augmented reality” or “AR” refers to systems where a user views images of the real world after they have passed through a computing system. For example, a tablet with a camera on the back can capture images of the real world and then display the images on the screen on the opposite side of the tablet from the camera. The tablet can process and adjust or “augment” the images as they pass through the system, such as by adding virtual objects. “Mixed reality” or “MR” refers to systems where light entering a user's eye is partially generated by a computing system and partially composes light reflected off objects in the real world. For example, a MR headset could be shaped as a pair of glasses with a pass-through display, which allows light from the real world to pass through a waveguide that simultaneously emits light from a projector in the MR headset, allowing the MR headset to present virtual objects intermixed with the real objects the user can see. “Artificial reality,” “extra reality,” or “XR,” as used herein, refers to any of VR, AR, MR, or any combination or hybrid thereof. Additional details on XR systems with which the disclosed technology can be used are provided in U.S. patent application Ser. No. 17/170,839, titled “INTEGRATING ARTIFICIAL REALITY AND OTHER COMPUTING DEVICES,” filed Feb. 8, 2021 and now issued as U.S. Pat. No. 11,402,964 on Aug. 2, 2022, which is herein incorporated by reference.

Those skilled in the art will appreciate that the components and blocks illustrated above may be altered in a variety of ways. For example, the order of the logic may be rearranged, substeps may be performed in parallel, illustrated logic may be omitted, other logic may be included, etc. As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc. Any patents, patent applications, and other references noted above are incorporated herein by reference. Aspects can be modified, if necessary, to employ the systems, functions, and concepts of the various references described above to provide yet further implementations. If statements or subject matter in a document incorporated by reference conflicts with statements or subject matter of this application, then this application shall control.

I/We claim:

1. A method for dynamically displaying three-dimensional web content to a user, the method comprising:
transmitting, by a browser in response to a received application programming interface (API) call from web code that implements a webpage, an indication that the browser and a XR system that implements the browser are configured to display three-dimensional content;
receiving a second API call from the web code that indicates the webpage comprises at least a first version and a second version, the versions supporting stereoscopic display the webpage; and
transitioning, based on the second API call, display of the webpage at the browser from a two-dimensional mode to a three-dimensional mode, wherein the three-dimensional mode causes an artificial reality system to render, via the browser, the first version of the webpage to a first eye of the user and the second version of the webpage to a second eye of the user, the rendering causing the user to perceive at least portions of the webpage as three-dimensional content.

2. A method for issuing decentralized entitlements by an issuing authority, the method comprising:
receiving a request for an entitlement from a user device, the entitlement being a right with respect to a resource;
issuing the entitlement as a token embedded with metadata specifying features of the entitlement;
signing the token with a private key associated with the issuing authority; and
transmitting the token to a messaging account associated with a user of the user device,
wherein the user device accesses the token via the messaging account and transmits the token to a validating authority different than the issuing authority, and
wherein the validating authority authenticates the token with a public key associated with the issuing authority and loads the entitlement using the metadata specifying the features of the entitlement.

3. A method for estimating energy expended by a user for artificial reality (XR) activity, the method comprising:
receiving pose data tracked for the user, wherein the pose data corresponds to upper and lower body movements of the user;
determining, using a kinematic model corresponding to the pose data, one or more changes in lower body posture of the user defined for respective changes in elevation between the user's head and a baseline;
estimating an energy expenditure associated with the one or more changes in lower body posture of the user, by:
applying a machine learning model to the determined one or more changes in lower body posture to obtain an estimated amount of energy expenditure due to the one or more changes in lower body posture of the user; and
estimating an overall energy expenditure of the user by combining an estimated amount of energy expenditure associated with the upper body movements of the user and the estimated amount of energy expenditure due to the one or more changes in lower body posture of the user.
